(19) United States
(12) Patent Application Publication
(10) Pub. No.: US 2024/0327570 A1
(43) Pub. Date: Oct. 3, 2024

(54) Electrocatalytic production of polyethylene furanoate degradable bioplastic

(71) Applicants: Yijin Kang, Naperville, IL (US); Ruya Zhang, Chengdu (CN)

(72) Inventors: Yijin Kang, Naperville, IL (US); Ruya Zhang, Chengdu (CN)

(21) Appl. No.: 18/199,029

(22) Filed: May 18, 2023

(30) Foreign Application Priority Data
Mar. 30, 2023 (CN) 1228870

(51) Int. Cl. C08G 63/16; C07D 307/68; C08G 63/83

(52) U.S. Cl. CPC C08G 63/16; C07D 307/68; C08G 63/83

(57) ABSTRACT

Herein, we enable a record-high FDCA reaction rate of 416 μmol h-1 cm-2 (1872 μmol h-1) at 100% FDCA selectivity using a ZnCo2O4 electrocatalyst in a near-neutral media that allows high concentration (i.e. 1M) of HMF, with assistance of an in-situ alkaline modification strategy that further enhances the reaction rate. In the light of the significantly improved FDCA production, we demonstrate production of PEF bioplastic from a biomass-derivative fructose, highly competitive to the petroleum-based production of PET plastics as indicated by our techno-economic analysis.

### CROSS-REFERENCE TO RELATED APPLICATIONS

This application relates back to CN 1,228,870 filed on Mar. 30, 2023 and is incorporated in its entirety.

### PRIOR ART

Plastics are a group of essential materials for human activities, that include transportation, construction, packaging, electronics, medicine, and etc., replacing their natural counterparts, for their low unit cost, versatility, temperature/water resistance, and etc¹. However, the growing demand for plastic products exacerbates the conflict between economic development and environmental protection².

Polyester feedstock used in the packaging market, which accounts for one-third of plastic demand, is synthesized by polymerization of petroleum-based chemicals, with great energy consumption in production³. As an example of popular polyester, polyethylene terephthalate (PET), is non-degradable, and can only be disposed of using landfill or incineration⁴.

### BACKGROUND

In contrast, biomass-based plastics (i.e. bioplastics) are usually sustainable, environmentally friendly, and biodegradable⁵. Among them, poly(ethylene 2,5-furandicarboxylate) (i.e. polyethylene furanoate, PEF) with its high glass transition temperature and great gas barrier properties, could be an ideal biomass-based substitute for the petroleum-based PET⁶.

2,5-furandicarboxylic acid (FDCA) is the key intermediate for production of PEF, just like petroleum-derived terephthalic acid (TPA) to PET. To compete with PET whose market size is ^{˜}$40 billion a year, PEF will be economically competitive only if the costs of FDCA can be significantly reduced⁷⁻⁸.

FDCA is primarily prepared by selective oxidation of 5-hydroxymethylfurfural (HMF). The conversion of HMF to FDCA can be achieved through homogeneous/heterogeneous catalysis⁹⁻¹⁰, photocatalysis¹¹, electrocatalysis¹²⁻¹⁶ and biological processes¹⁷⁻¹⁸. In industry, the HMF-to-FDCA conversion is usually achieved through thermocatalysis, in alkaline solution (pH>13), medium to high temperature (30-140° C.), and high oxygen pressure (0.3-2.0 MPa)¹⁹⁻²⁰.

Recently, researchers proposed an electrocatalytic route for producing FDCA^{13,21-24} that has a number of advantages over thermocatalysis, including simple devices and mild conditions to effectively reduce energy consumption, no need for additional oxidant due to the reactive oxygen species produced by electro-activation of H₂O, ready coupling with value-added half-reaction such as hydrogen evolution reaction (HER) to subsidize the production of FDCA, and etc²⁵.

To date, numerous technologies and catalysts have been suggested for the electrocatalytic production of FDCA. However, most of the reported electrocatalytic processes for HMF-to-FDCA conversion were carried out in a strong alkaline media, where HMF undergoes significant dimerization and other side reactions²⁶⁻²⁹ (FIGS. 6 and 7), resulting demonstrations only in low HMF concentration (e.g. 0-20 mmol/L) which has no industrial value. Low reactant concentrations not only increase energy input, but also increase separation costs exponentially with reduced product concentrations³⁰.

Although a few cases have been demonstrated at low pH where the high reactant concentration is allowed^{27,31}, the substantially reduced reaction rate brings competition from the formation of by-products such as aldehydes that leads to the loss of FDCA yield³²⁻³³. Therefore, in order to enable any industrial application, the problem of low reactant (i.e. HMF) concentration and low product (i.e. FDCA) yield has to be addressed.

Herein, we offer an activated ZnCo₂O₄ nanoarray as the anodic catalysts for HMF electrooxidation in a near neutral media (i.e. KHCO₃), achieving 100% conversion of HMF and 99% selectivity of FDCA. Furthermore, high FDCA product rate (416 μmol h⁻¹ cm⁻²) with high FDCA Faradaic efficiency (>90%) is achieved in flow cell by a strategy of “in-situ alkaline modification” that enables a high concentration HMF (^{˜}1 mol/L). With our new technology that enables scalable production of FDCA from HMF, we design a protocol for production of PEF bioplastic from biomass-derivatives such as fructose. Preliminary techno-economic analysis (TEA) estimates net revenues of around $200/ton FDCA by our electrochemical process from fructose to PEF under a commercially relevant current density (i.e. 100 mA cm⁻²). Herein, we offer a viable and profitable pathway to produce environment-friendly PEF bioplastics.

### Pathway Design and Techno-Economic Analysis

The key factor for industrial PEF production is an efficient and economic FDCA monomer production. Compared with the traditional thermochemical oxidation to prepare FDCA, the electro-oxidation route can achieve more accurate and convenient control of the product selectivity and reaction rate, and the equipment for the electrocatalytic process is simple and inexpensive to manufacture, set up, and maintain³⁴.

Furthermore, in order to solve the bottleneck problem of FDCA electro-synthesis limited by low reaction concentration of HMF otherwise the inability of HMF leading to loss of reactant, we render an in-situ alkaline modification strategy to provide sufficient OH⁻ to facilitate the reaction while precisely controlling the pH not to trigger the polymerization and other side reactions of the reactants, and therefore achieving a high rate of FDCA production in a near-neutral media that is desirable for industry.

Moreover, we propose to optimize the production process of HMF, the reactant for FDCA production. The typical biomass pathway production of HMF via dehydration of hexose requires using dimethyl sulfoxide (DMSO) as co-solvent, or organic catalytic systems (e.g. in ionic liquids)¹⁹, usually associating with high cost equipment for separation and purification as well as possible pollution from the used organic compounds³⁵.

In contrast, we identify diphenyl sulfoxide (DPhSO), a low-freezing organic catalyst, and we only need one simple extraction to pass the produced HMF solution to the electrolyzer feeding³⁶.

### SUMMARY OF THE INVENTION

In this context, we demonstrate an integrated process for production of PEF bioplastic with a side-product of clean fuel (H₂) from fructose, as shown in FIG. 1a. The whole process is composed of three steps: (i) dehydrogenation of fructose to produce HMF, (ii) a near-neutral electro-oxidation of HMF to FDCA, paired with hydrogen production, (iii) PEF production.

Compared to conventional routes^{19,37-39} and other previously reported electrocatalytic route³⁴, our route reduces overall cost from five ways: reduced cost in reactants; improved energy efficiency; optimized reaction rates that is usable for industry; less product separation and purification steps; and simple and inexpensive equipment. The economic feasibility of such design is demonstrated in a techno-economic analysis (FIG. 1b) using a simplified model adapted from literatures⁴⁰⁻⁴¹.

The analysis results suggest that the market price of FDCA depends on renewable electricity costs, Faradaic efficiency to FDCA (FE_{FDCA}) and current density (FIG. 7). Only if the price of FDCA is controlled below 100% to 150% of the market price of TPA ($1445/ton)⁷, PER may have the potential to replace PET in the consumer market⁴².

At the current density of 50 mA cm 2, the FDCA sale price is $1346.9/ton when renewable electricity cost is controlled at 10 cents kWh-1 and the catalyst achieves sufficiently high FE_{FDCA} (i.e. 85%). The FDCA sale price can be further reduced to $1219.6/ton when the current density is increased to 100 mA cm⁻².

Techno-economic analysis indicates that the potential of PEF in the plastic market and industry depends on Faradaic efficiency of HMF-to-FDCA and useful current density, which has not been achieved by any available technology, to the best of our knowledge. Here, we are delighted to introduce our approach to realize the industrial application of PEF production from fructose, enabled by advanced electrocatalyst and novel in-situ alkaline modification technique.

### The Advanced Electrocatalyst for Neutral HMF Electro-Oxidation

We identify a spinel activate-ZnCo₂O₄ catalyst with high-valence Co rich surface that exhibits excellent HMF electro-oxidation performance with a record-high FE_{FDCA} in neutral conditions. Pristine-ZnCo₂O₄ supported on nickel foam was synthesized using a hydrothermal process and a subsequent calcination.

The pristine-ZnCo₂O₄ displays a three-dimensional interconnected array structure with uniform nanowires (FIG. 9). To obtain the activated-ZnCo₂O₄ catalyst, the pristine-ZnCo₂O₄ was electrochemically activated under constant voltage of 1.7 V in 1 M KHCO₃ electrolyte for 10000 s (FIG. 10).

The powder X-ray diffraction (XRD) patterns of the as-synthesized and activated-ZnCo₂O₄ catalyst indicate a spinel structure of ZnCo₂O₄, showing no phase transition after the activation process (PDF 23-1390, FIG. 2h), also confirmed by selected area electron diffraction (SAED, FIGS. 11 and 12). Transmission electron microscope (TEM) images show that both the pristine-ZnCo₂O₄ and the activated-ZnCo₂O₄ catalysts exhibit similar nanowire morphology (FIG. 2a-b, 2d-e). The element distribution was investigated by energy dispersive X-ray spectroscopy (EDS) mapping. The leaching of zinc during activation was also revealed by the EDS (FIG. 2c, 2f, and Table S1). Inductively coupled plasma atomic emission spectroscopy (ICP-AES) also detected substantial amount of Zn in the electrolyte, confirming the leaching of Zn during the activation process (Table S2).

To investigate the surface valence change induced by the activation process, we performed X-ray photoelectron spectroscopy (XPS). As shown in FIG. 2i, after activation, the ratio of Co³⁺:Co²⁺ on the surface of catalyst increased from 1.69 to 2.66, indicating more high valence Co sites were exposed on the surface of activated-ZnCo₂O₄. As the control, similar Co₃O₄ nanowires were also synthesized and fully characterized (FIG. 13-17).

### Electrochemical Characterization

The electrochemical HMF oxidation was investigated in a near-neutral electrolyte (1 M KHCO₃) with 10 mM HMF in a standard three-electrode H-cell and quantified by high performance liquid chromatography (HPLC).

To facilitate comparison, we present the product distribution and Faradaic efficiency (FE_{FDCA}) at 60 C, 90 C, 120 C (note: charge for fully oxidizing 10 mM HMF in 10 mL electrolyte is 57.89 C, theoretically) to investigate the electrocatalytic HMF oxidation performance on different catalysts.

The electrocatalytic performances of the activated-ZnCo₂O₄, Co₃O₄, and the pristine-ZnCo₂O₄ were explored (FIG. 18-22). Co₃O₄, an extensively studied alkaline HMF electro-oxidation catalyst⁴³, showed descent HMF oxidation performance compared to nickel foam in a near-neutral electrolyte (FIG. 23). In 1 M KHCO₃ electrolyte at 1.6 V, FDCA selectivity on Co₃O₄ reached 34.4%, 66.9%, 86.4% at 60 C, 90 C, 120 C, respectively, but only with FE_{FDCA} no more than 43.3%. Pristine-ZnCo₂O₄ in which tetrahedral Co atoms in the spinel structure are replaced by Zn atoms (FIG. 2g), exhibited better performance than Co₃O₄, reaching the FDCA yield of 38.7%, 78.9%, 96.5% at 60 C, 90 C, 120 C, respectively, as well as a maximal FE_{FDCA} of 50.7%.

In the spinel cobalt oxides, the Co³⁺ in octahedral CoO₆ is believed to be the active site for the reaction involving oxygen-containing intermediates⁴⁴. The electrochemically activated ZnCo₂O₄ in which leaching of surface Zn atoms in tetrahedral site enabled surface enrichment of Co³⁺, and thus exhibited excellent HMF oxidation performance⁴³. In KHCO₃ electrolyte at applied voltage of 1.6 V, activated-ZnCo₂O₄ showed over 97% HMF conversion with 45.4%2-formyl-5-furancarboxylic acid (FFCA) yield and 51.5% FDCA yield at 60 C (FIG. 3a). With the increase of charge, the ≈100% HMF conversion, ≈99% FDCA selectivity are achieved with maximum 57.8% FE_{FDCA} (FIG. 3b, S19c). During a durability test that ran for 6 successive cycles, the great stability of ZnCo₂O₄ catalyst was confirmed as the FDCA selectivity was well maintained at ^{˜}98% (FIG. 3d). Overall, the activated-ZnCo₂O₄ demonstrated a significant improvement in catalytic performance toward neutral HMF oxidation to FDCA, compared to previously reported electrocatalysts³²⁻³³.

As shown in FIG. 3a, the whole reaction process of neutral HMF oxidation can be divided into two parts. The first one is the complete conversion of HMF to various oxidation products such as FFCA and FDCA; the second one is the full oxidation of partial oxidation products such as FFCA to FDCA. Unlike in alkaline electrolyte (e.g. KOH) to produce FDCA, in neutral conditions HMF oxidation prefers to produce aldehyde product FFCA over the completely oxidized acid product FDCA, like other alcohol oxidation reactions^{32,45-46}.

In neutral conditions the primary competing reaction to the oxidation of HMF is the oxygen evolution reaction (OER), which is responsible for the reduced FE_{FDCA} and becomes more dominant at higher voltage. For instance, using activated-ZnCo₂O₄ electrocatalyst with 10 mM HMF, as the voltage increased from 1.5 V to 1.7 V, the charge required for complete conversion of HMF to FDCA increased from 100 C to over 130 C, and the maximum FE_{FDCA} decreased from 66.2% to 46.3% (FIGS. 25 and 26).

Increasing concentration of HMF is an effective way to suppress the OER competition. For instances, the reaction with 1000 mM HMF exhibited a reduced overpotential (1.568 V at 50 mA/cm²) by 104 mV to that with 10 mM HMF (1.672 V at 50 mA/cm²) (FIG. 27); at higher HMF concentration, the required charge for full conversion of HMF-to-FDCA got closer to the theoretical coulomb value and the FE_{FDCA} increased substantially to 84.5% (FIGS. 3c, S23, and S24). In the near-neutral electrolyte, high HMF concentration is allowed, and thus the desirable FE_{FDCA} is enabled.

### In-Situ Alkaline Modification

In neutral HMF oxidation reaction, using activated-ZnCo₂O₄ electrocatalyst, we achieve 100% FDCA selectivity with up to 84.5% FE_{FDCA} at 100 mM HMF concentration. However, the low FDCA production rate greatly limits further industrial production of PEF (Table S3).

The HMF oxidation occurs on hydroxyl groups and aldehyde groups that are attached to the furan ring. The product distribution during the reaction indicates that FDCA is generated via the route of HMF-DFF (2,5-diformylfuran)-FFCA-FDCA in neutral electrolyte where OH⁻ species are consumed during each step (FIG. 4a). Furthermore, the concentrated alkaline solution (e.g., 10M KOH) is added immediately before the reactant flows into the electrolyzer instead of adding alkaline in the stock solution of the reactant as has been done in the past. The pH of the outlet electrolyzer is subsequently monitored to verify it remains between a pH of 7 and 11.

The first deprotonation step in electrocatalytic alcohol oxidation reaction is a base-catalyzed process to generate active intermediate⁴⁵, while the similar case was reported for aldehyde oxidation reaction⁴⁶. During the reaction, due to the consumption of OH⁻ in each step, limited OH⁻ in the near-neutral electrolyte depletes quickly, resulting in a rapid decrease in the reaction rate (FIG. 30). Maintaining sufficient OH⁻ supply at the electrode surface during the reaction, but not triggering the side-reaction of HMF, required a delicate balance to achieve a high rate HMF electrooxidation. Accordingly, we employ an in-situ alkaline modification strategy to dose alkaline solutions based on the OH⁻ consumption rate into the HMF electrooxidation to enable FDCA production with a high rate that is practical to plastic industry.

The in-situ alkaline modification strategy was demonstrated in the electrochemical HMF oxidation on the activated-ZnCo₂O₄ catalyst in 1 M KHCO₃ electrolyte with 100^{˜}1000 mM HMF in a flow-cell. As shown in FIG. 4b, at 1.6 V with 100 mM HMF, a FDCA production rate of 74.5 μmol h⁻¹ cm⁻² was achieved, which is a 17-fold enhancement compared to the original process (3.6 μmol h⁻¹ cm⁻²) without an alkaline modification (FIG. 31). Combining the in-situ alkaline modification and 1M HMF reactant, a record-high FDCA production rate of 416 μmol h⁻¹ cm 2 is achieved (FIG. 4c, 32, Table S4). Carbon balance and pH monitoring proves that the alkaline modification neither leads to a loss of high concentration HMF nor side reactions (FIG. 33).

### From Fructose to PEF Plastic

In the light of the significantly improved FDCA production, we are able to demonstrate the production of H₂ and PEF bioplastic from biomass-derivative fructose (FIGS. 5a and 34). As the first step, the dehydrogenation reaction is carried out at 140° C. with fructose and DPhSO as feedstock and catalyst, respectively.

After the reaction, due to the insolubility of DPhSO in water and the high partition coefficient of HMF in the DPhSO/water, the HMF product can be simply collected by extraction³⁶ (FIGS. 35 and 36), and the obtained HMF solution can be directly fed to the flow-cell electrolyzer with KHCO₃. In the electrolyzer, the activated-ZnCo₂O₄ is used as the anode catalyst for HMF oxidation, and a nickel foam is used as the cathode catalyst for hydrogen evolution.

With the in-situ alkaline modification (by KOH), the HMF solution flow is converted into a flow of FDCA solution under applied potential (FIG. 37). Then, the FDCA solution is acidified by HCl and thus the FDCA precipitates as a solid, which is subsequently separated and collected as white powder. The composition and purity of FDCA product are confirmed by ¹H NMR and ¹³C NMR spectra (FIGS. 5b, 5c, and 38). The obtained FDCA is used as feedstock to produce PEF bioplastic via a two-step condensation polymerization⁴⁷⁻⁴⁸. The final product of PEF is presented in a form of plastic pellet (FIGS. 5d, 5e, and 39), at a scale as large as up to kilo-gram. Due to the simple process, production of PEF bioplastic from biomass-derived fructose can be readily scaled-up, as a pilot scale test is being carried out.

The degradable PEF bioplastics made from biomass-derivatives are highly desirable for the consideration of environment and sustainability. However, for a long time, the high cost of FDCA has been limiting the competition of PEF to the petroleum-derived PET plastics.

Electrocatalytic production of FDCA was proposed, however, has been disallowed for any practical industrial application due to the limitation of reactant concentration in alkaline media otherwise the sluggish reaction in neutral media.

Herein, we first time circumvent both low reaction rate in neutral conditions and side-reaction of HMF at high concentration in alkaline media by a highly efficient ZnCo₂O₄ electrocatalyst that can be used in a near-neutral electrolyte, and an in-situ alkaline modification strategy that boosts the reaction rate of HMF oxidation while keeping the side-reaction of HMF under control.

While a full picture from fructose to PEF bioplastic has been demonstrated, our techno-economic analysis indicates that the cost of FDCA can be reduced to $1139/ton (based on current density of 80 mA cm⁻², best FEFDCA of 97%, and best local electricity cost of ^{˜}7 [symbol] /kWh), fueling the competition over PET plastic where TPA costs $1445/ton.

### DESCRIPTION OF DRAWINGS

FIG. 1. Biomass to degradable bioplastics. (a) Illustration of the concept. (b) Breakdown of costs at current densities of 50 and 100 mA/cm², as calculated from a techno-economic analysis. Calculation details are provided in the supplementary text and Figure.S3.

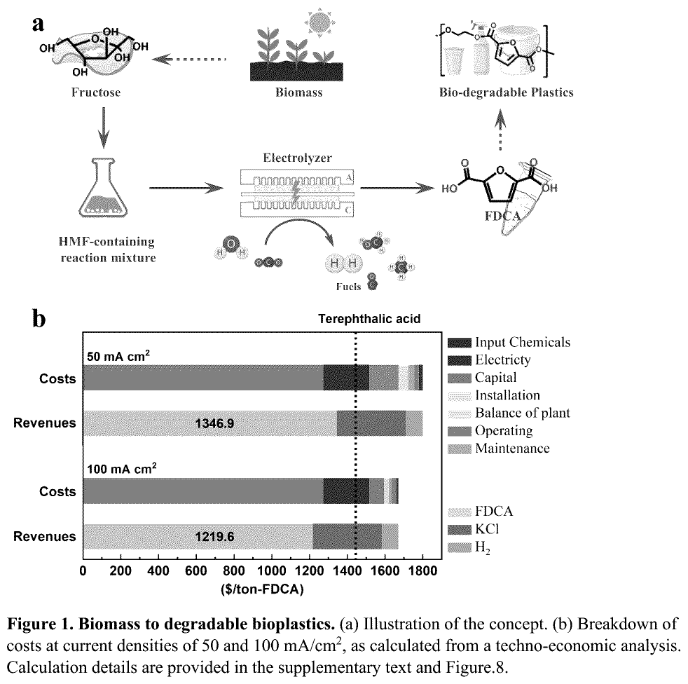

FIG. 2. Advanced ZnCo₂O₄ electrocatalysts. (a, b) TEM images (inset of b: SEM image), (c) EDS mapping of the pristine-ZnCo₂O₄. (d, e) TEM images, (f) EDS mapping for activated-ZnCo₂O₄. (g) Crystal structure of ZnCo₂O₄. (h) XRD patterns, (i) Co 2p of XPS spectra of the pristine-ZnCo₂O₄ and the activated-ZnCo₂O₄.

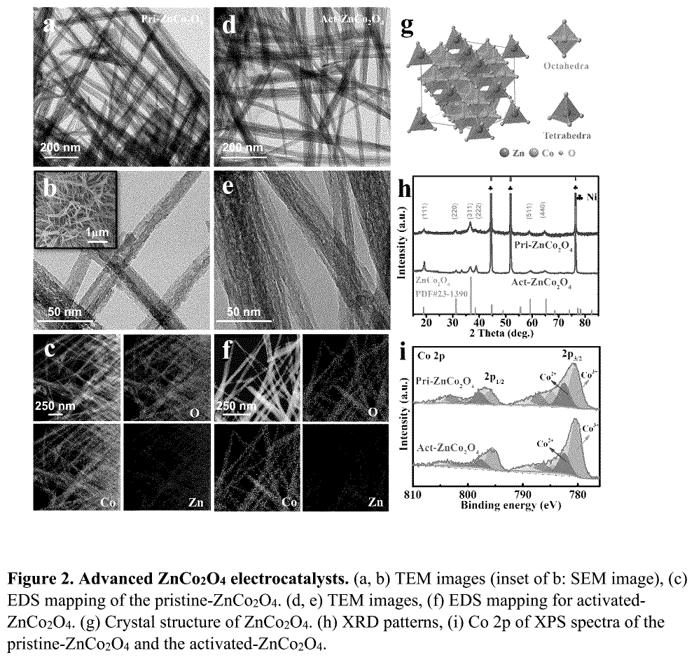

FIG. 3. Catalytic Performance of the activated-ZnCo₂O₄ in 1 M KHCO₃. (a) Conversion (%) of HMF and yield (%) of the oxidation products with 10 mM HMF at 1.6 V. (b) The bar chart of product yield (%) with 10 mM HMF at different voltages. (c) FDCA yield (%) with different concentration of HMF at 1.6 V. (d) HMF conversion (%), FDCA selectivity (%), and Faradaic efficiency (%) of FDCA for six successive cycles with 10 mM HMF at 1.6 V.

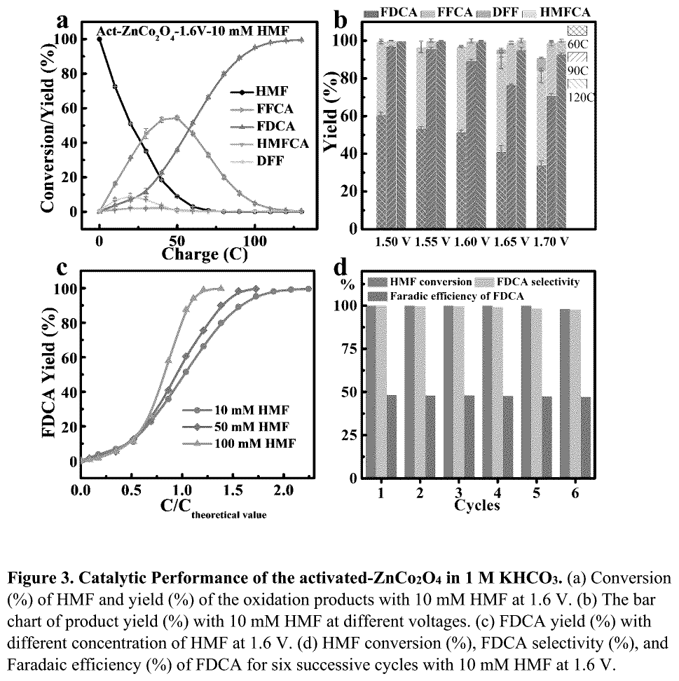

FIG. 4. In-situ Alkaline Modification strategy. (a) Illustration of a nucleophilic oxidation reaction mechanism for electrochemical oxidation of HMF. (b) Current vs. time and FDCA selectivity vs. time for reactions with and without the in-situ alkaline modification. (c) Comparison of the catalytic performances of activated-ZnCo₂O₄ with the in-situ alkaline modification strategy and other systems reported in literatures^{15,25,33}.

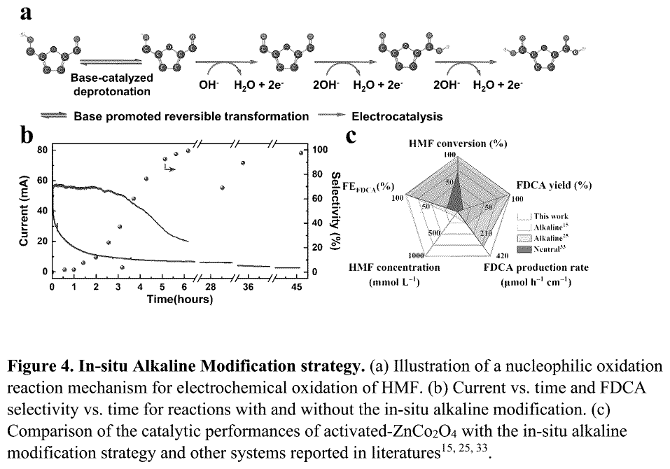

FIG. 5. Fructose to PEF plastics. (a) The flow diagram of the process from biomass-derivative fructose to PEF bioplastics. (b) Photo and (c) ¹H NMR analysis of the FDCA product. (d) Photo and (e) ¹H NMR analysis of the PEF product.

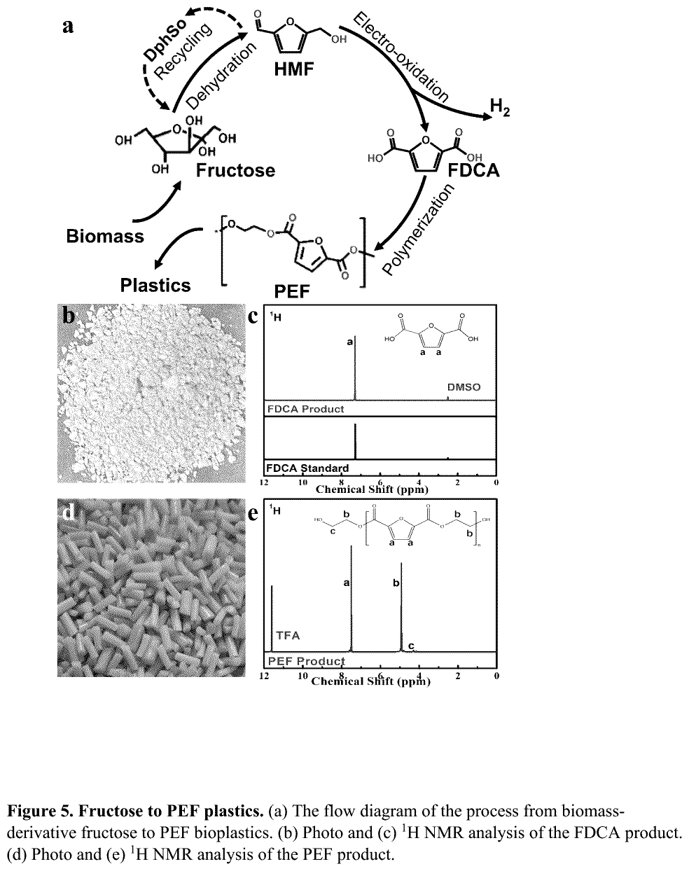

FIG. 6. (a) Comparison of HMF retention (%) in different electrolytes with 10 mM and 100 mM HMF added. (b) HMF concentration over time in 1 M KHCO₃.

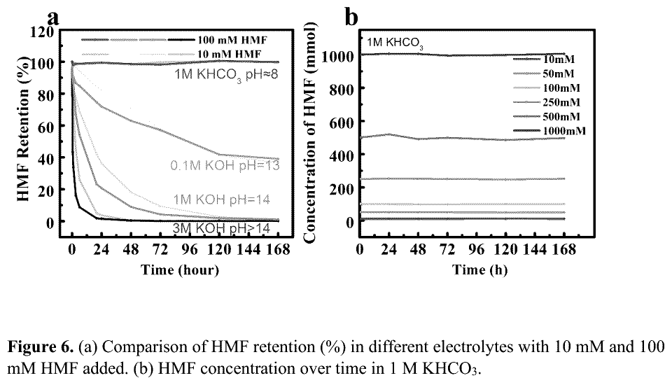

FIG. 7. Color change over time, showing the degree of side reaction for the HMF in different concentration and in different electrolytes.

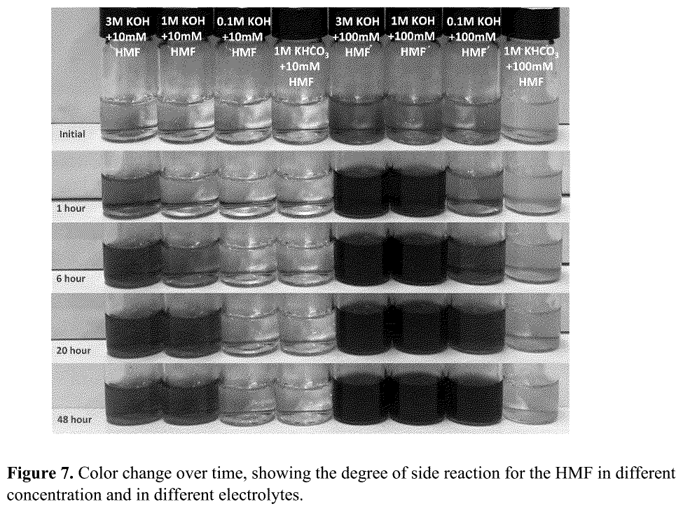

FIG. 8. Techno-economic analysis of FDCA production showing plant-gate levelized cost as a function of Faradaic efficiency to FDCA (FE_{FDCA}), renewable electricity cost, and current density. (a) 50 mA cm⁻², (b) 100 mA cm⁻², (c) 300 mA cm⁻².

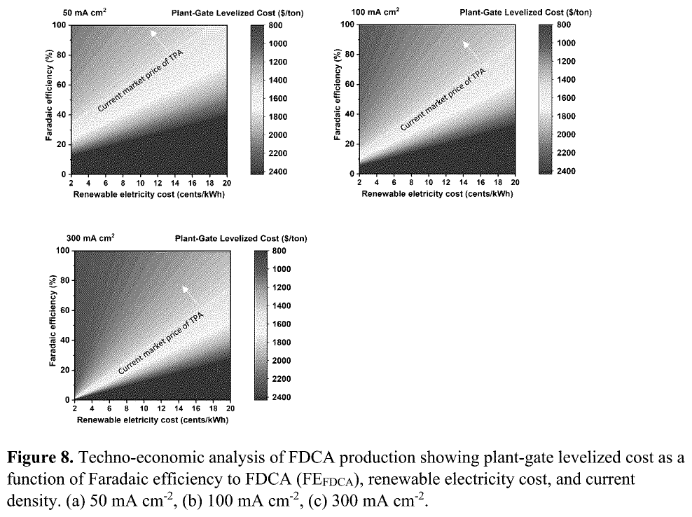

FIG. 9. SEM images of the pristine-ZnCo₂O₄.

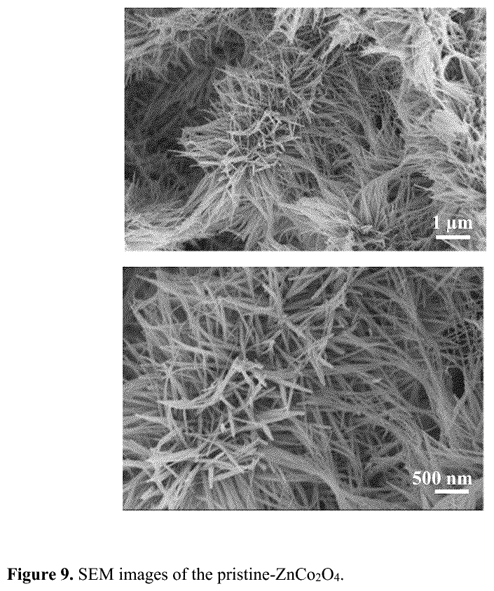

FIG. 10. i-t curves of activation process of ZnCo₂O₄.

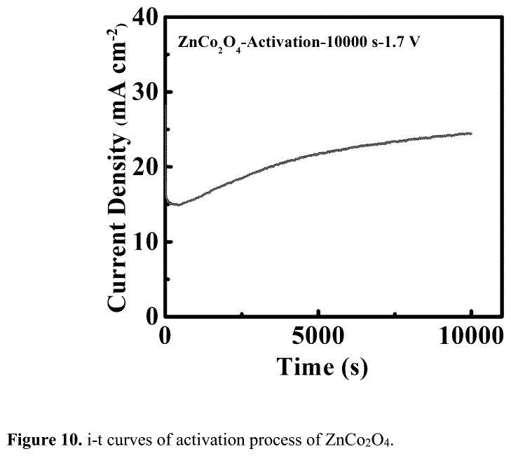

FIG. 11. (a) HRTEM image and (b) SAED pattern of the pristine-ZnCo₂O₄.

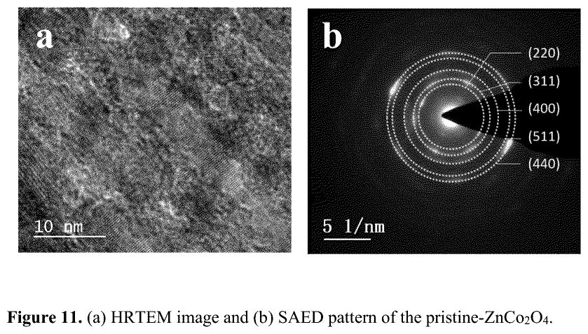

FIG. 12. (a) HRTEM image and (b) SAED pattern of the activated-ZnCo₂O₄.

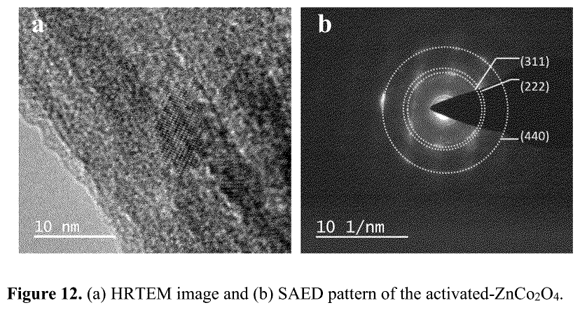

FIG. 13. SEM images of Co₃O₄.

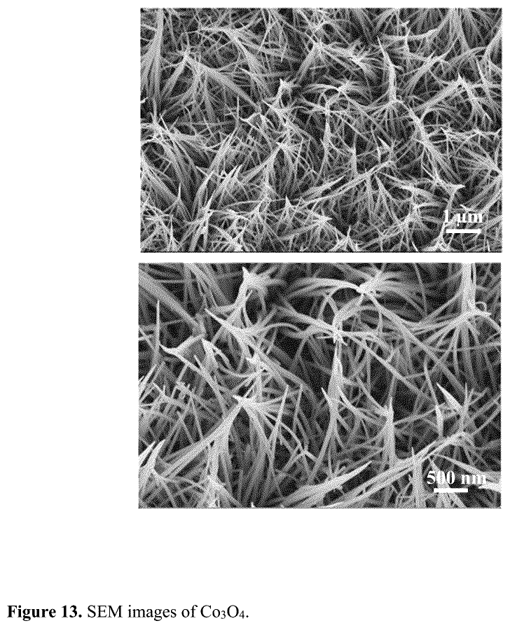

FIG. 14. (a, b) TEM images, (c) HRTEM image, and (d) SAED pattern of Co₃O₄.

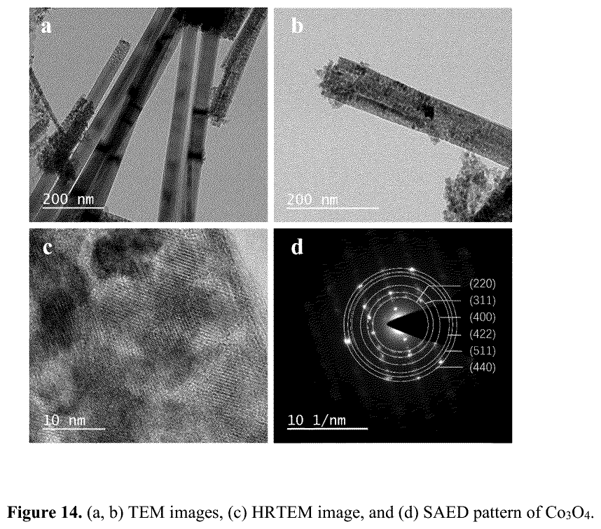

FIG. 15. EDS mapping of Co₃O₄.

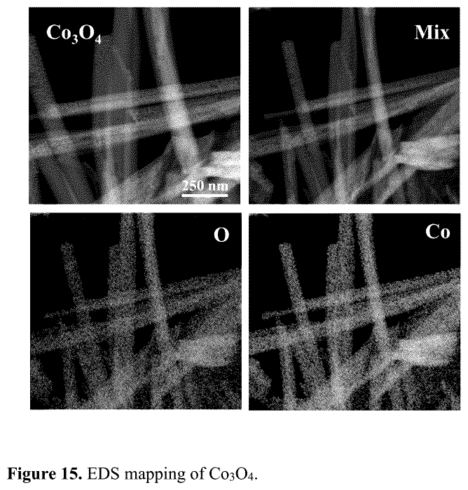

FIG. 16. XRD pattern of Co₃O₄.

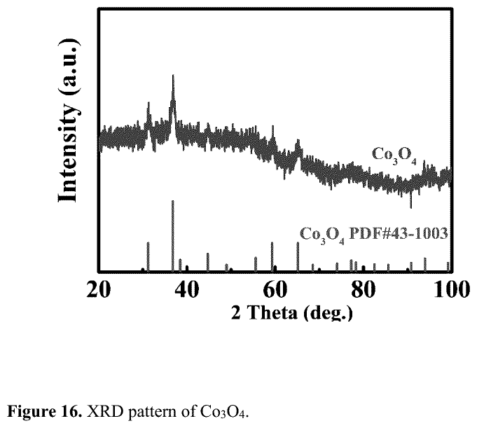

FIG. 17. Co 2p XPS spectrum of Co₃O₄.

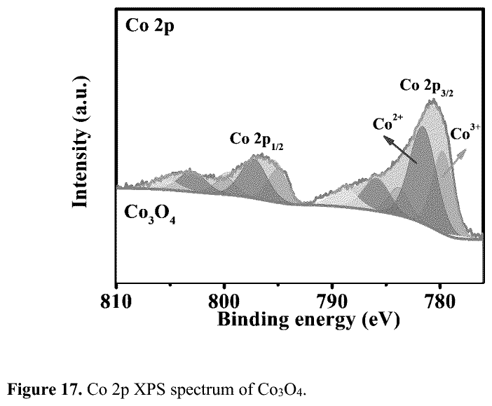

FIG. 18. (a-d) Conversion (%) of HMF and yield (%) of oxidation products catalyzed by Co₃O₄ in 1 M KHCO₃ with 10 mM HMF at different voltages (1.5 V, 1.55 V, 1.6 V, 1.65 V). (e) Bar chart of product yield on Co₃O₄ electrocatalyst at different voltages with 10 mM HMF.

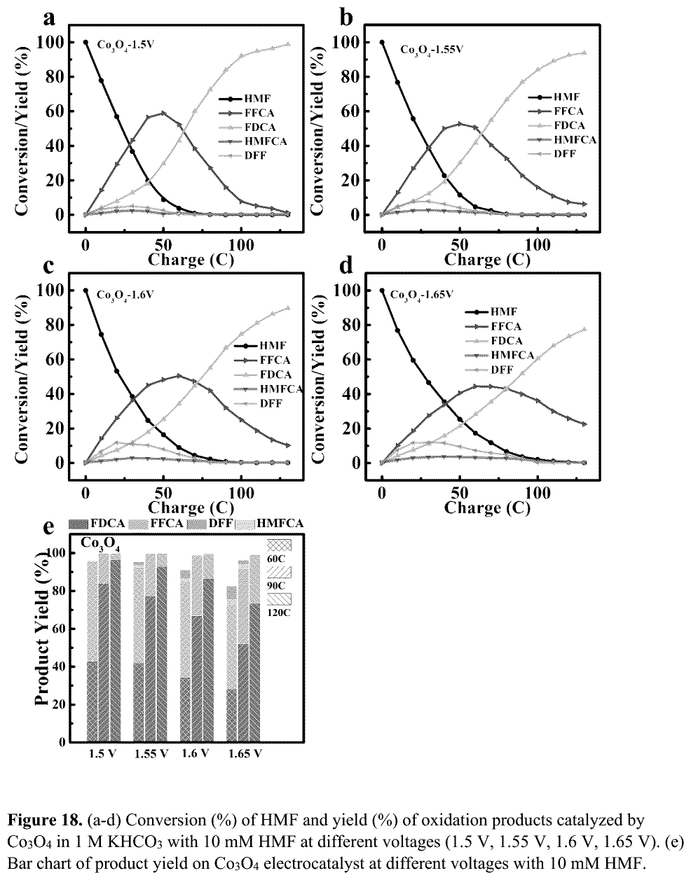

FIG. 19. Faradaic efficiency catalyzed by Co₃O₄ in 1 M KHCO₃ with 10 mM HMF at different voltages (1.5 V, 1.55 V, 1.6 V, 1.65 V).

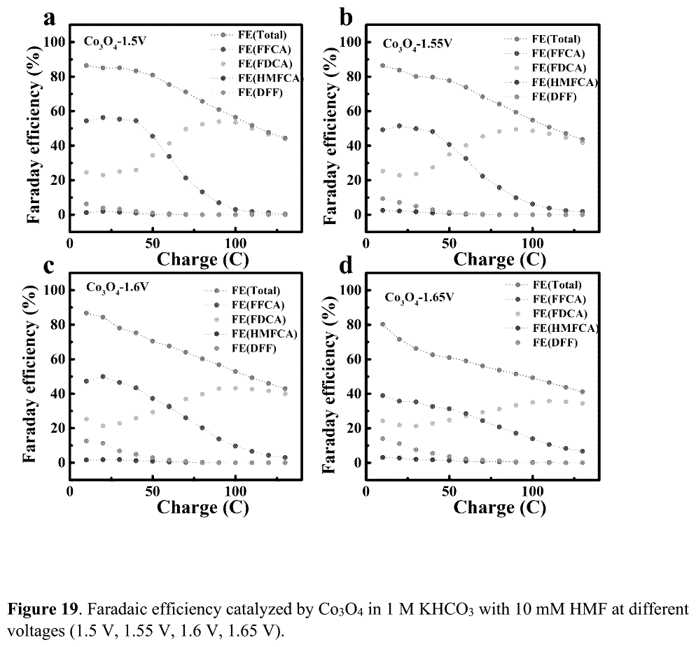

FIG. 20. (a-e) Conversion (%) of HMF and yield (%) of oxidation products catalyzed by the pristine-ZnCo₂O₄ in 1 M KHCO₃ with 10 mM HMF at different voltages (1.5 V, 1.55 V, 1.6 V, 1.65 V, 1.7 V). (f) Bar chart of product yield on the pristine-ZnCo₂O₄ electrocatalyst at different voltages with 10 mM HMF.

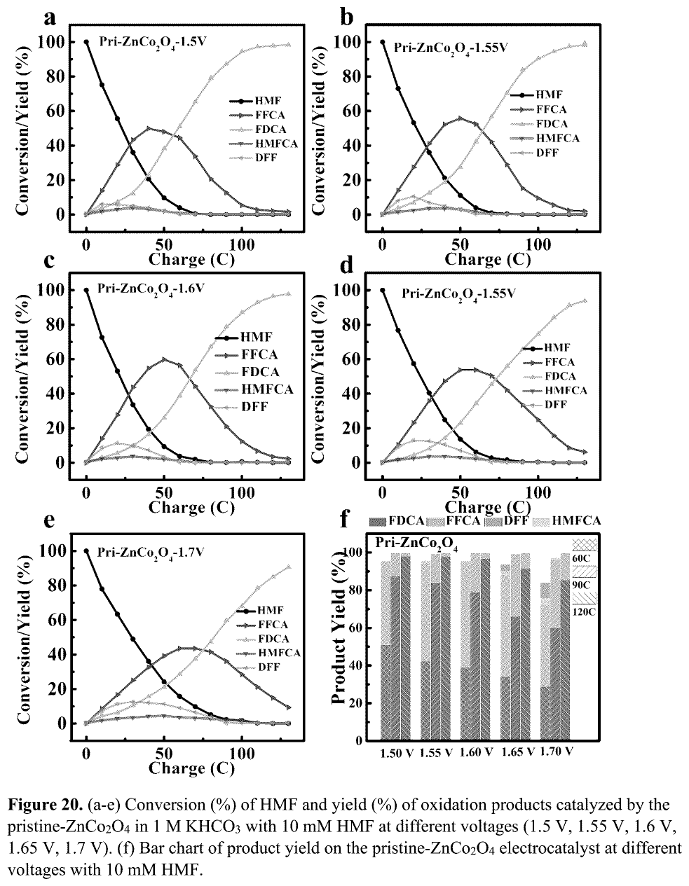

FIG. 21. Faradaic efficiency catalyzed by the pristine-ZnCo₂O₄ in 1 M KHCO₃ with 10 mM HMF at different voltages (1.5 V, 1.55 V, 1.6 V, 1.65 V, 1.7 V).

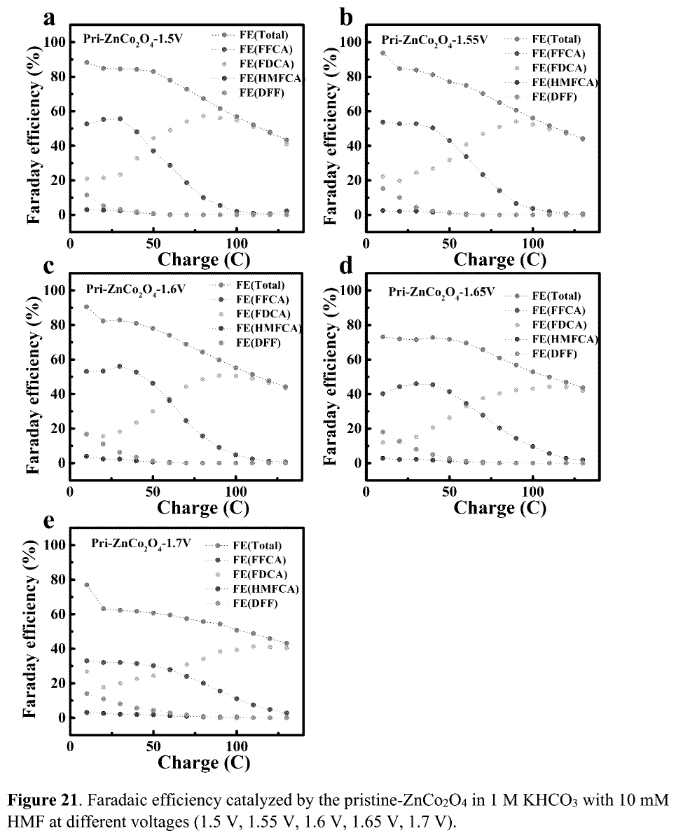

FIG. 22. (a) Bar chart of product yield (%), (b) FDCA yield (%) with 10 mM HMF at 1.6 V catalyzed by Co₃O₄, pristine-ZnCo₂O₄ and activated-ZnCo₂O₄.

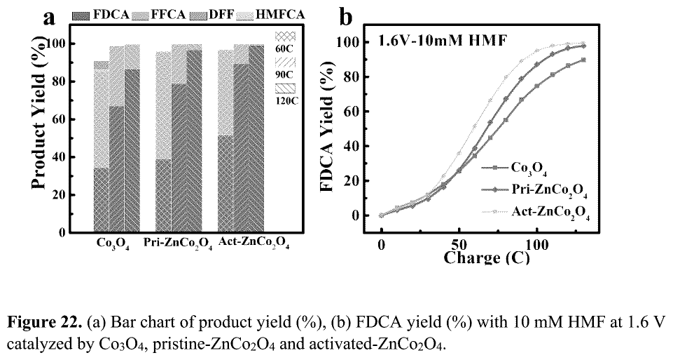

FIG. 23. Conversion (%) of HMF and yield (%) of oxidation products catalyzed by Ni foam in 1 M KHCO₃ with 10 mM HMF at 1.6 V.

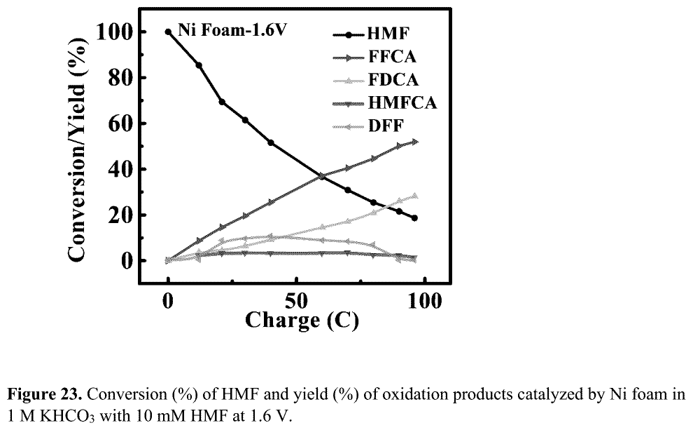

FIG. 24. Faradaic efficiency catalyzed by the activated-ZnCo₂O₄ in 1 M KHCO₃ with 10 mM HMF at different voltages (1.5 V, 1.55 V, 1.6 V, 1.65 V, 1.7 V).

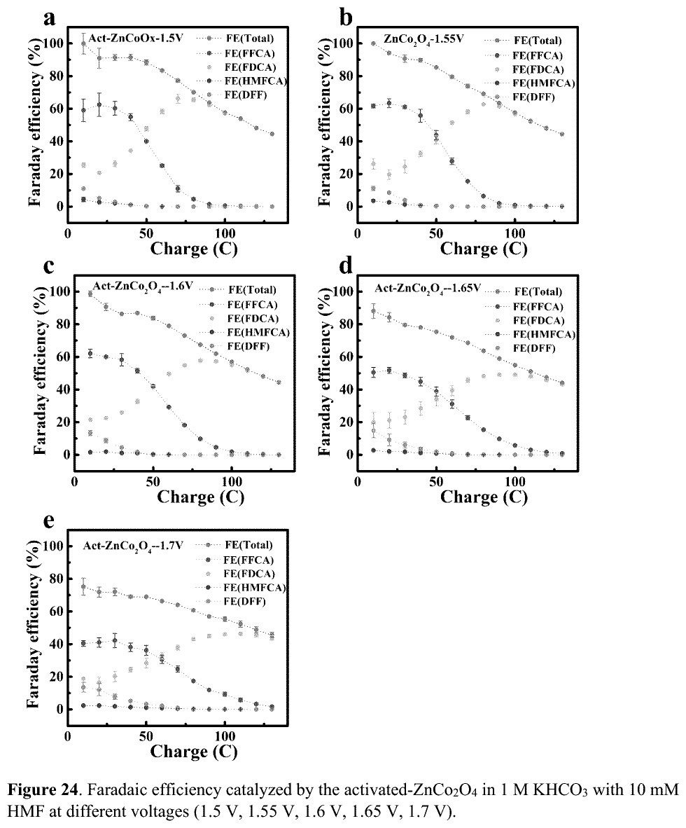

FIG. 25. Conversion (%) of HMF and yield (%) of oxidation products catalyzed by the activated-ZnCo₂O₄ in 1 M KHCO₃ with 10 mM HMF at different voltages (1.5 V, 1.55 V, 1.65 V, 1.7 V).

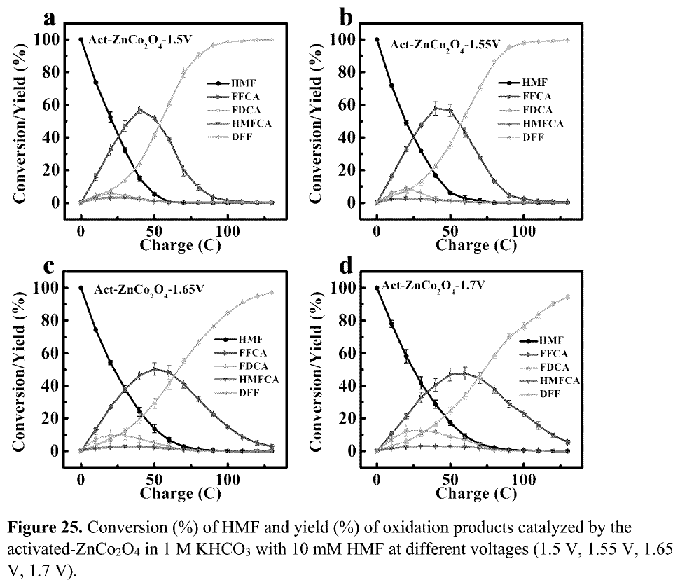

FIG. 26. Total Faradaic efficiency and Faradaic efficiency of FDCA catalyzed by the activated-ZnCo₂O₄ in 1 M KHCO₃ with 10 mM HMF at different voltages (1.5 V, 1.55 V, 1.6 V, 1.65 V, 1.7 V).

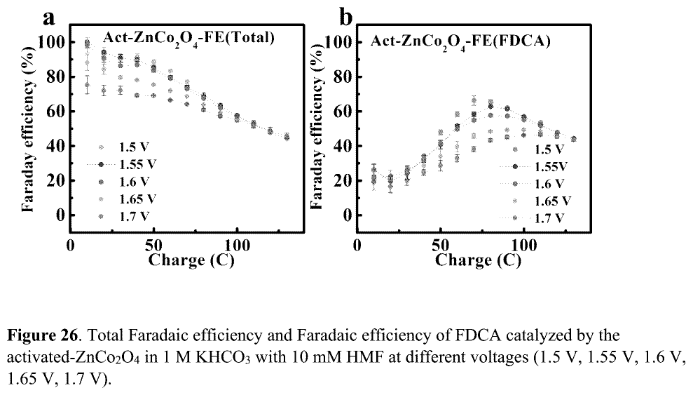

FIG. 27. Linear sweep voltammetry curves of HMF oxidation on the activated-ZnCo₂O₄ electrocatalyst with HMF at different concentrations in 1 M KHCO₃.

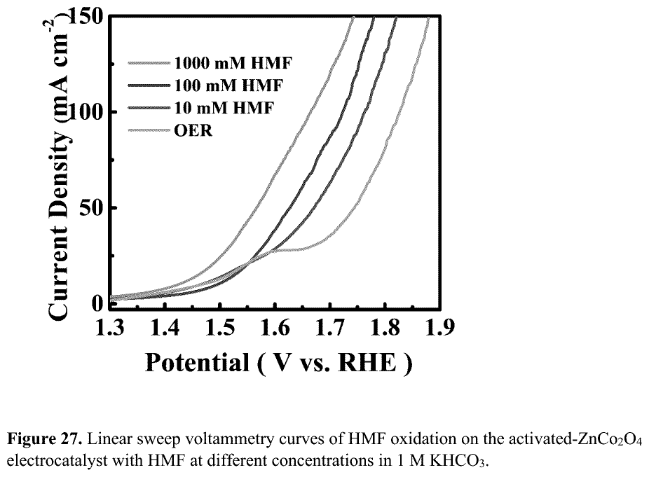

FIG. 28. Conversion of HMF and yield of oxidation products catalyzed by the activated-ZnCo₂O₄ in 1 M KHCO₃ at 1.6 V with (a) 50 mM HMF, (b) 100 mM HMF.

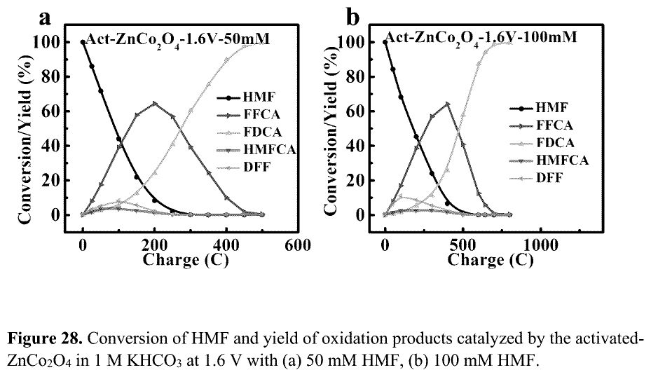

FIG. 29. (a, b) Faradaic efficiency of various products on the activated-ZnCo₂O₄ in 1 M KHCO₃ with different HMF concentrations at 1.6 V. (c) Faradaic efficiency of FDCA vs. HMF concentration (10 mM, 50 mM, 100 mM).

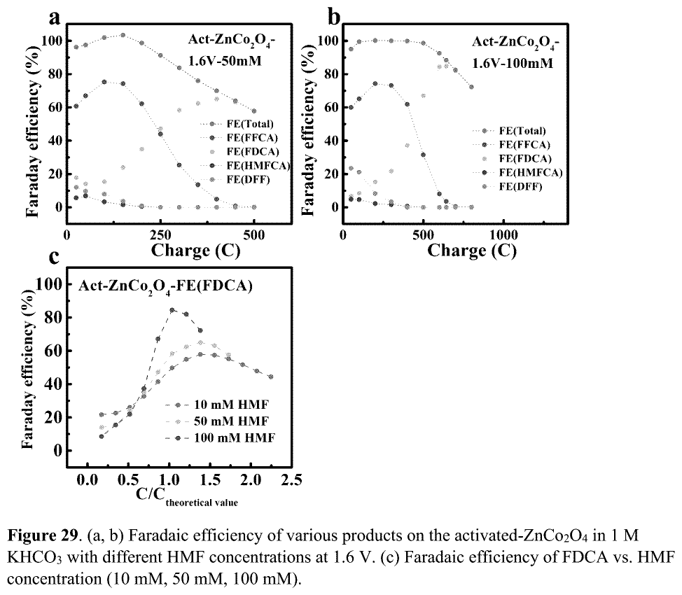

FIG. 30. Current density vs. time and charge vs. time curves taken on the activated-ZnCo₂O₄ at 1.6 V with 10 mM HMF.

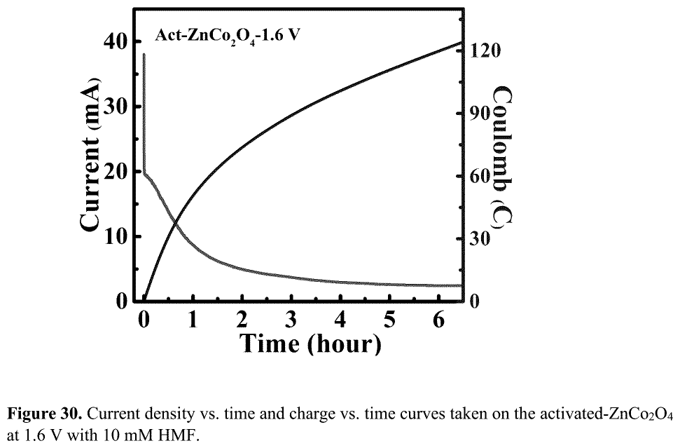

FIG. 31. (a) Conversion (%) of HMF and yield (%) of oxidation products, (b) Faradaic efficiency of products on the activated-ZnCo₂O₄ in 1 M KHCO₃ with 100 mM HMF at 1.7 V, with the in-situ alkaline modification.

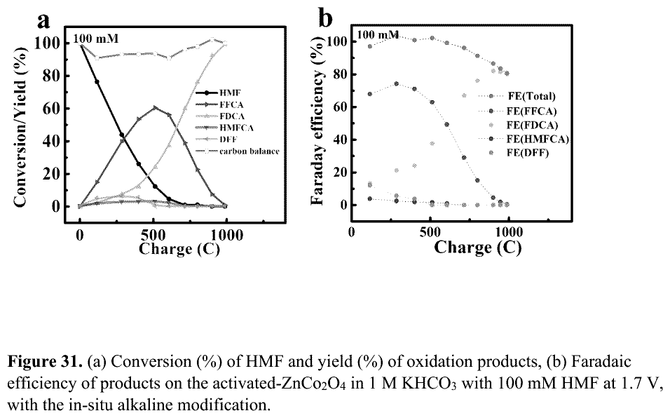

FIG. 32. Comparison of the catalytic performances. FDCA production rate over various catalysts listed on Supplementary Table S4. Note: in the upper chart, some reports used overly small area for reaction, making an unfair comparison for the specific production rate directly.

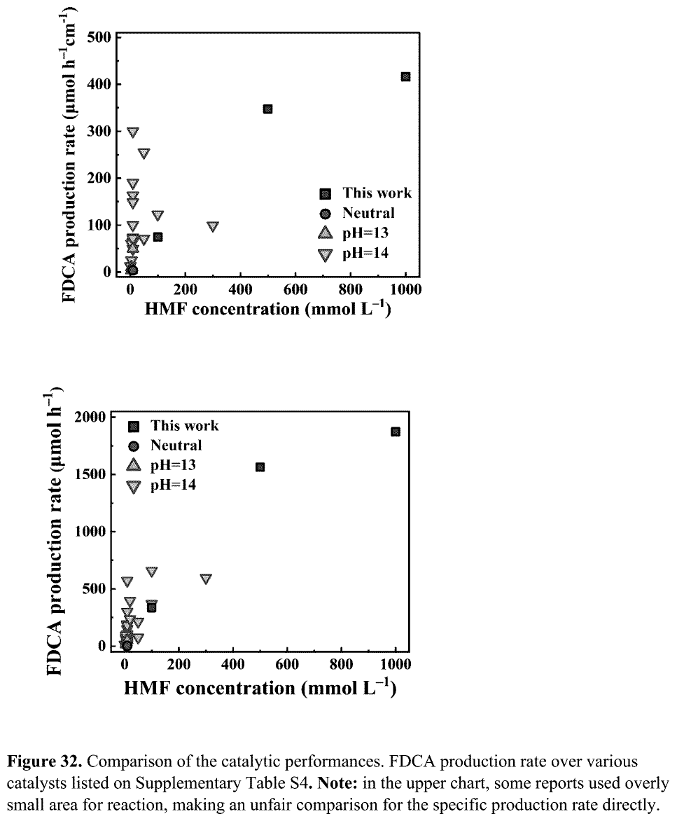

FIG. 33. (a) Conversion (%) of HMF and yield (%) of oxidation products, (b) pH and carbon balance change curves, (c) Faradaic efficiency, and (d) current density vs. time and charge vs. time curves (on the activated-ZnCo₂O₄ in 1 M KHCO₃ with 1000 mM HMF at 1.7 V with the in-situ alkaline modification).

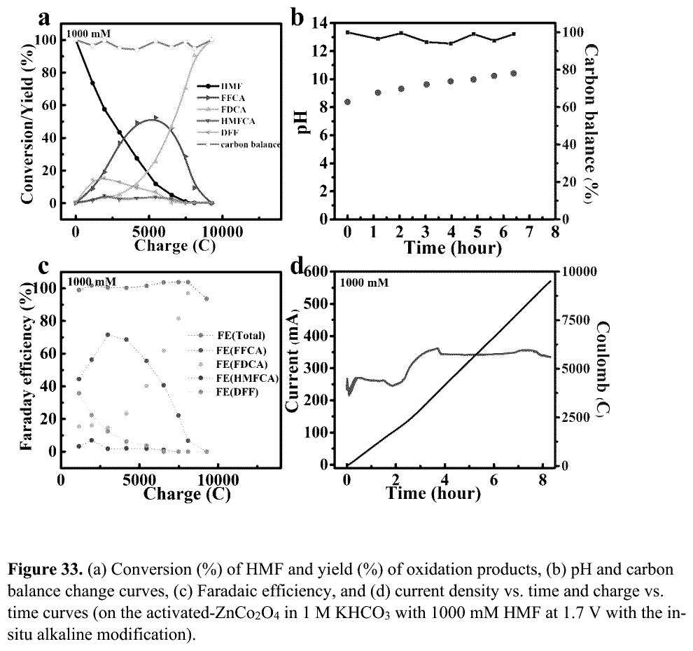

FIG. 34. Schematic diagram of the FDCA product synthesis from fructose.

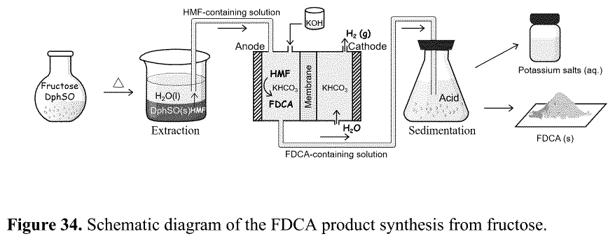

FIG. 35. Photo of (a) extraction and (b) fructose-derived HMF solution.

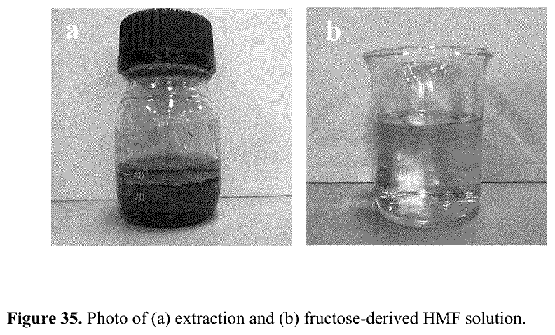

FIG. 36. ¹H NMR analysis of fructose derived HMF.

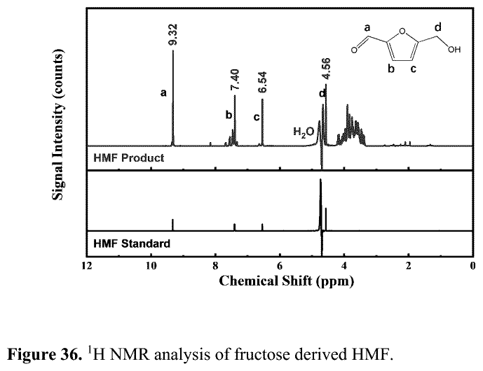

FIG. 37. (a) Conversion (%) of HMF and yield (%) of oxidation products. (b) Current density vs. time and charge vs. time (catalyzed by activated-ZnCo₂O₄, 1 M KHCO₃ with fructose-derived HMF, at 1.7 V, with alkaline modification).

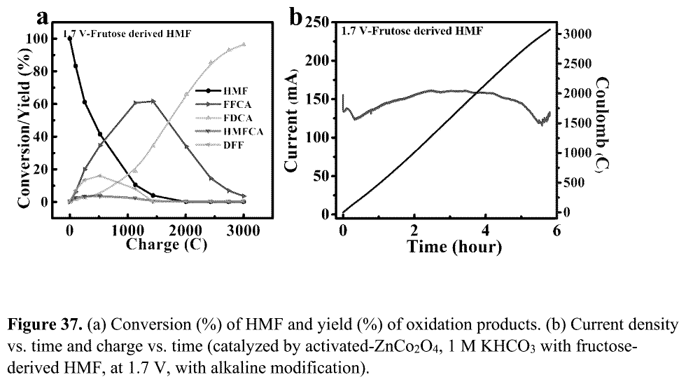

FIG. 38. ¹³C NMR analysis of the synthesized FDCA.

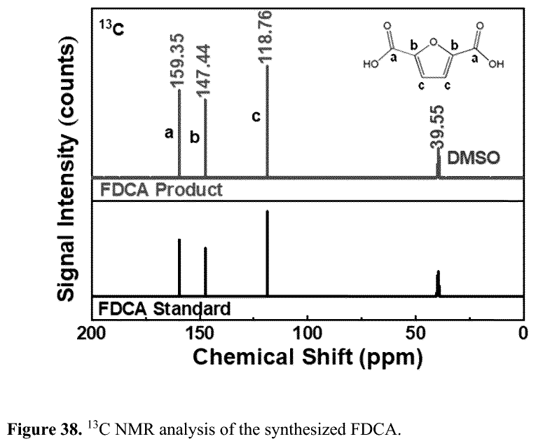

FIG. 39. ¹³C NMR analysis of PEF plastic.

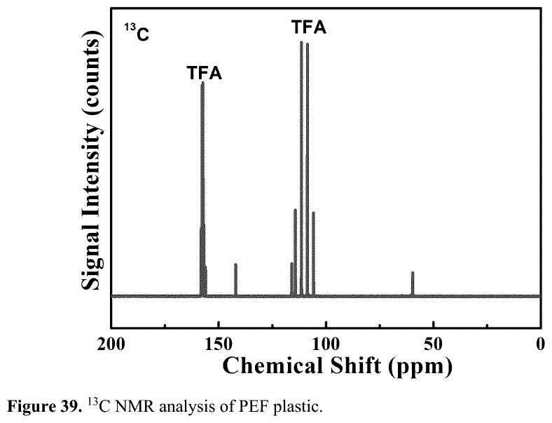

### REFERENCES

1. Choi, S.; Balamurugan, M.; Lee, K.-G.; Cho, K. H.; Park, S.; Seo, H.; Nam, K. T., Mechanistic investigation of biomass oxidation using nickel oxide nanoparticles in a CO₂-saturated electrolyte for paired electrolysis. J. Phys. Chem. Lett. 2020, 11 (8), 2941-2948.
2. Taitt, B. J.; Nam, D. H.; Choi, K. S., A comparative study of nickel, cobalt, and iron oxyhydroxide anodes for the electrochemical oxidation of 5-hydroxymethylfurfural to 2,5-furandicarboxylic acid. ACS Catal. 2019, 9 (1), 660-670.
3. Chen, J.; Wang, Y.; Zhou, M.; Li, Y., Boosting the electro-oxidation of 5-hydroxymethyl-furfural on a Co—CoSₓ heterojunction by intensified spin polarization. Chem. Sci. 2022, 13 (16), 4647-4653.
4. Cai, M.; Zhang, Y.; Zhao, Y.; Liu, Q.; Li, Y.; Li, G., Two-dimensional metal-organic framework nanosheets for highly efficient electrocatalytic biomass 5-(hydroxymethyl)furfural (HMF) valorization. J. Mater. Chem. A 2020, 8 (39), 20386-20392.
5. Zhang, R.; Jiang, S.; Rao, Y.; Chen, S.; Yue, Q.; Kang, Y., Electrochemical biomass upgrading on CoOOH nanosheets in a hybrid water electrolyzer. Green Chem. 2021, 23 (6), 2525-2530.
6. Kong, F.; Wang, M., Preparation of sulfur-modulated nickel/carbon composites from lignosulfonate for the electrocatalytic oxidation of 5-hydroxymethylfurfural to 2,5-furandicarboxylic acid. ACS Appl. Energy Mater. 2021, 4 (2), 1182-1188.
7. Liu, W. J.; Dang, L. N.; Xu, Z. R.; Yu, H. Q.; Jin, S.; Huber, G. W., Electrochemical oxidation of 5-hydroxymethylfurfural with NiFe layered double hydroxide (LDH) nanosheet catalysts. ACS Catal. 2018, 8 (6), 5533-5541.
8. Lu, Y.; Liu, T.; Huang, Y.-C.; Zhou, L.; Li, Y.; Chen, W.; Yang, L.; Zhou, B.; Wu, Y.; Kong, Z.; Huang, Z.; Li, Y.; Dong, C.-L.; Wang, S.; Zou, Y., Integrated catalytic sites for highly efficient electrochemical oxidation of the aldehyde and hydroxyl groups in 5-hydroxymethylfurfural. ACS Catal. 2022, 12 (7), 4242-4251.
9. Pang, X.; Bai, H.; Zhao, H.; Fan, W.; Shi, W., Efficient electrocatalytic oxidation of 5-hydroxymethylfurfural coupled with 4-nitrophenol hydrogenation in a water system. ACS Catal. 2022, 12 (2), 1545-1557.
10. Qi, Y.-F.; Wang, K.-Y.; Sun, Y.; Wang, J.; Wang, C., Engineering the electronic structure of NiFe layered double hydroxide nanosheet array by implanting cationic vacancies for efficient electrochemical conversion of 5-hydroxymethylfurfural to 2,5-furandicarboxylic acid. ACS Sustainable Chem. Eng. 2022, 10 (1), 645-654.
11. Zhu, B.; Qin, Y.; Du, J.; Zhang, F.; Lei, X., Ammonia etching to generate oxygen vacancies on CuMn₂O₄ for highly efficient electrocatalytic oxidation of 5-hydroxymethylfurfural. ACS Sustainable Chem. Eng. 2021, 9 (35), 11790-11797.
12. Li, S.; Sun, X.; Yao, Z.; Zhong, X.; Coo, Y.; Liang, Y.; Wei, Z.; Deng, S.; Zhuang, G.; Li, X.; Wang, J., Biomass valorization via paired electrosynthesis over vanadium nitride-based electrocatalysts. Adv. Funct. Mater. 2019, 29 (42), 1904780.
13. Sun, Y.; Wang, J.; Qi, Y.; Li, W.; Wang, C., Efficient electrooxidation of 5-hydroxymethylfurfural using Co-doped Ni₃S₂ catalyst: Promising for H₂ production under industrial-level current density. Adv. Sci. 2022, 9 (17), 2200957.
14. You, B.; Jiang, N.; Liu, X.; Sun, Y. J., Simultaneous H₂ generation and biomass upgrading in water by an efficient noble-metal-free bifunctional electrocatalyst. Angew. Chem. Int. Ed. 2016, 55 (34), 9913-9917.
15. Lu, X.; Wu, K.-H.; Zhang, B.; Chen, J.; Li, F.; Su, B.-J.; Yan, P.; Chen, J.-M.; Qi, W., Highly efficient electro-reforming of 5-hydroxymethylfurfural on vertically oriented nickel nanosheet/carbon hybrid catalysts: Structure-function relationships. Angew. Chem. Int. Ed. 2021, 60 (26), 14528-14535.
16. Chen, C.; Zhou, Z.; Liu, J.; Zhu, B.; Hu, H.; Yang, Y.; Chen, G.; Gao, M.; Zhang, J., Sustainable biomass upgrading coupled with H₂ generation over in-situ oxidized Co₃O₄ electrocatalysts. Appl. Catal., B 2022, 307, 121209.
17. Gao, L.; Liu, Z.; Ma, J.; Zhong, L.; Song, Z.; Xu, J.; Gan, S.; Han, D.; Niu, L., NiSe@NiOx core-shell nanowires as a non-precious electrocatalyst for upgrading 5-hydroxymethylfurfural into 2,5-furandicarboxylic acid. Appl. Catal., B 2020, 261, 118235.
18. Kang, M. J.; Park, H.; Jegal, J.; Hwang, S. Y.; Kang, Y. S.; Cha, H. G., Electrocatalysis of 5-hydroxymethylfurfural at cobalt based spinel catalysts with filamentous nanoarchitecture in alkaline media. Appl. Catal., B 2019, 242, 85-91.
19. Wang, W.; Wang, M., Nitrogen modulated NiMoO₄ with enhanced activity for the electrochemical oxidation of 5-hydroxymethylfurfural to 2,5-furandicarboxylic acid. Catal. Sci. Technol. 2021, 11 (22), 7326-7330.
20. Wang, D.; Gong, W.; Zhang, J.; Wang, G.; Zhang, H.; Zhao, H., In situ growth of MOFs on Ni(OH)₂ for efficient electrocatalytic oxidation of 5-hydroxymethylfurfural. Chem. Commun. 2021, 57 (86), 11358-11361.
21. Wang, C.; Bongard, H.-J.; Yu, M.; Schuth, F., Highly ordered mesoporous Co₃O₄ electrocatalyst for efficient, selective, and stable oxidation of 5-hydroxymethylfurfural to 2,5-furandicarboxylic acid. ChemSusChem 2021, 14 (23), 5199-5206.
22. Wang, W.; Kong, F.; Zhang, Z.; Yang, L.; Wang, M., Sulfidation of nickel foam with enhanced electrocatalytic oxidation of 5-hydroxymethylfurfural to 2,5-furandicarboxylic acid. Dalton Trans. 2021, 50 (31), 10922-10927.
23. Huang, X.; Song, J.; Hua, M.; Xie, Z.; Liu, S.; Wu, T.; Yang, G.; Han, B., Enhancing the electrocatalytic activity of CoO for the oxidation of 5-hydroxymethylfurfural by introducing oxygen vacancies. Green Chem. 2020, 22 (3), 843-849.
24. Zhang, Y.; Xue, Z.; Zhao, X.; Zhang, B.; Mu, T., Controllable and facile preparation of CO₉S₈—Ni₃S₂ heterostructures embedded with N,S,O-tri-doped carbon for electrocatalytic oxidation of 5-hydroxymethylfurfural. Green Chem. 2022, 24 (4), 1721-1731.
25. Wang, H.; Li, C.; An, J.; Zhuang, Y.; Tao, S., Surface reconstruction of NiCoP for enhanced biomass upgrading. J. Mater. Chem. A 2021, 9 (34), 18421-18430.
26. Bai, X.-J.; He, W.-X.; Lu, X.-Y.; Fu, Y.; Qi, W., Electrochemical oxidation of 5-hydroxymethylfurfural on ternary metal-organic framework nanoarrays: enhancement from electronic structure modulation. J. Mater. Chem. A 2021, 9 (25), 14270-14275.
27. Deng, X.; Kang, X.; Li, M.; Xiang, K.; Wang, C.; Guo, Z.; Zhang, J.; Fu, X.-Z.; Luo, J.-L., Coupling efficient biomass upgrading with H₂ production via bifunctional CuxS@NiCo-LDH core-shell nanoarray electrocatalysts. J. Mater. Chem. A 2020, 8 (3), 1138-1146.
28. Zheng, L.; Zhao, Y.; Xu, P.; Lv, Z.; Shi, X.; Zheng, H., Biomass upgrading coupled with H₂ production via a nonprecious and versatile Cu-doped nickel nanotube electrocatalyst. J. Mater. Chem. A 2022, 10 (18), 10181-10191.
29. Zhang, J.; Yu, P.; Zeng, G.; Bao, F.; Yuan, Y.; Huang, H., Boosting HMF oxidation performance via decorating ultrathin nickel hydroxide nanosheets with amorphous copper hydroxide islands. J. Mater. Chem. A 2021, 9 (15), 9685-9691.
30. Yang, S.; Guo, Y.; Zhao, Y.; Zhang, L.; Shen, H.; Wang, J.; Li, J.; Wu, C.; Wang, W.; Cao, Y.; Zhuo, S.; Zhang, Q.; Zhang, H., Construction of synergistic Ni₃S₂—MoS₂ nanoheterojunctions on Ni foam as bifunctional electrocatalyst for hydrogen evolution integrated with biomass valorization. Small 2022, 18 (24), 2201306.
31. Leow, W. R.; Lum, Y.; Ozden, A.; Wang, Y.; Nam, D.-H.; Chen, B.; Wicks, J.; Zhuang, T.-T.; Li, F.; Sinton, D.; Sargent, E. H., Chloride-mediated selective electrosynthesis of ethylene and propylene oxides at high current density. Science 2020, 368 (6496), 1228-1233.
32. De Luna, P.; Hahn, C.; Higgins, D.; Jaffer, S. A.; Jaramillo, T. F.; Sargent, E. H., What would it take for renewably powered electrosynthesis to displace petrochemical processes? Science 2019, 364 (6438), eaav3506.
33. Massaro, M. C.; Monteverde, A. H. A., Techno-economic analysis of FDCA production through electrocatalytic processes. J. Electrochem. Soc. 2022, 169 (5), 054515.
34. Zhou, H.; Ren, Y.; Li, Z.; Xu, M.; Wang, Y.; Ge, R.; Kong, X.; Zheng, L.; Duan, H., Electrocatalytic upcycling of polyethylene terephthalate to commodity chemicals and H₂ fuel. Nat. Commun. 2021, 12 (1), 4679.
35. Liu, W.-J.; Xu, Z.; Zhao, D.; Pan, X.-Q.; Li, H.-C.; Hu, X.; Fan, Z.-Y.; Wang, W.-K.; Zhao, G.-H.; Jin, S.; Huber, G. W.; Yu, H.-Q., Efficient electrochemical production of glucaric acid and H₂ via glucose electrolysis. Nat. Commun. 2020, 11 (1), 265.

### Supplementary Tables

TABLE S1
The atomic ratio of Co, Zn of the pristine ZnCo₂O₄
and the activated-ZnCo₂O₄ based on the EDS mapping.

| Samples | Co:Zn |
|---|---|
| Pri-ZnCo₂O₄ | 4.19:1 |
| Act-ZnCo₂O₄ | 4.97:1 |

TABLE S2
The content of Co and Zn in electrolyte after
activation process based on ICP results.

| | Concentration of Co | Concentration of Zn |
|---|---|---|
| Samples | (mg/L) | (mg/L) |
| Electrolyte after activation | 1.22 | 0.72 |
| process | | |

TABLE S3
Summary of the HMF conversion, FDCA selectivity, Faradaic
efficiency, and production rate of FDCA by the activated-ZnCo₂O₄ in
1M KHCO₃ with various HMF concentration at 1.6 V.

| HMF | | | | | |
|---|---|---|---|---|---|
| concen- | | HMF | FDCA | FE | Production |
| tration | Time | Conversion | Selectivity | (FDCA) | rate of FDCA |
| (mM/L) | (h) | (%) | (%) | (%) | (μmol h⁻¹ cm⁻¹) |
| 10 | 6 | 100 | >99.5% | 57 | 3.7 |
| 50 | 21 | 100 | >99.5% | 62 | 5.2 |
| 100 | 45 | 100 | >99.5% | 84 | 4.7 |

1. A method of electrocatalytic production of polyethylene furanoate degradable bioplastic comprising of dehydrogenation of fructose to produce HMF, a near-neutral electro-oxidation of HMF to FDCA, pair with hydrogen production, and PEF production.

2. The method of electrocatalytic production of polyethylene furanoate degradable bioplastic comprising of dehydrogenation of fructose to produce HMF, near-neutral electro-oxidation of HMF to FDCA, pair with hydrogen production, and PEF production as in claim 1, wherein a spinel activated-ZnCo₂O₄ catalyst with high-valence Co rich surface that exhibits excellent HMF electro-oxidation performance with a record-high FE_{FDCA} in neutral conditions is used.

3. The method of electrocatalytic production of polyethylene furanoate degradable bioplastic comprising of dehydrogenation of fructose to produce HMF, near-neutral electro-oxidation of HMF to FDCA, pair with hydrogen production, and PEF production as in claim 1, wherein pristine-ZnCo₂O₄ supported on nickel foam was synthesized using a hydrothermal process and a subsequent calcination.

4. The method of electrocatalytic production of polyethylene furanoate degradable bioplastic comprising of dehydrogenation of fructose to produce HMF, near-neutral electro-oxidation of HMF to FDCA, pair with hydrogen production, and PEF production as in claim 1, wherein to obtain the activated-ZnCo₂O₄ catalyst, the pristine-ZnCo₂O₄ was electrochemically activated under constant voltage of 1.7 V in 1 M KHCO₃ electrolyte for 10000 s.

5. A method of in-situ alkaline modification of maintaining OH⁻ supply at the electrode surface during the reaction, but not triggering the side-reaction of HMF comprising of an in-situ alkaline modification strategy to dose alkaline solutions based on OH⁻ consumption rate into the HMF electrooxidation to enable FDCA production with a high rate that is practical.

6. The method of in-situ alkaline modification of maintaining OH⁻ supply at the electrode surface during the reaction, but not triggering the side-reaction of HMF comprising of an in-situ alkaline modification strategy to dose alkaline solutions based on OH⁻ consumption rate into the HMF electrooxidation to enable FDCA production with a high rate that is practical as in claim 5, wherein activated-ZnCo₂O₄ electrocatalyst is used to achieve 100% FDCA with up to 84.5% FE_{FDCA} at 100 mM HMF concentration.

7. The method of in-situ alkaline modification of maintaining OH⁻ supply at the electrode surface during the reaction, but not triggering the side-reaction of HMF comprising of an in-situ alkaline modification strategy to dose alkaline solutions based on OH⁻ consumption rate into the HMF electrooxidation to enable FDCA production with a high rate that is practical as in claim 5, wherein the electrochemical HMF oxidation on the activated-ZnCo₂O₄ catalyst in 1 M KHCO₃ electrolyte with 100^{˜}1000 mM HMF in a flow-cell.

8. The method of in-situ alkaline modification of maintaining OH⁻ supply at the electrode surface during the reaction, but not triggering the side-reaction of HMF comprising of an in-situ alkaline modification strategy to dose alkaline solutions based on OH⁻ consumption rate into the HMF electrooxidation to enable FDCA production with a high rate that is practical as in claim 5, wherein a 17-fold enhancement compared to the original process (3.6 μmol h⁻¹ cm⁻²) without an alkaline modification is achieved at 1.6 V with 100 mM HMF, a FDCA production rate of 74.5 μmol h⁻¹ cm⁻².

9. The method of in-situ alkaline modification of maintaining OH⁻ supply at the electrode surface during the reaction, but not triggering the side-reaction of HMF comprising of an in-situ alkaline modification strategy to dose alkaline solutions based on OH⁻ consumption rate into the HMF electrooxidation to enable FDCA production with a high rate that is practical as in claim 5, wherein combining the in-situ alkaline modification and 1M HMF reactant, a record-high FDCA production rate of 416 μmol h⁻¹ cm⁻² is achieved without a loss of high concentration HMF nor side reactions.

10. The method of electrocatalytic production of polyethylene furanoate degradable bioplastic comprising of dehydrogenation of fructose to produce HMF, the near-neutral electro-oxidation of HMF to FDCA, pair with hydrogen production, and PEF production as in claim 1, wherein the dehydrogenation reaction is carried out at 140° C. with fructose and DPhSO as feedstock and catalyst, respectively.

11. The method of electrocatalytic production of polyethylene furanoate degradable bioplastic comprising of dehydrogenation of fructose to produce HMF, the near-neutral electro-oxidation of HMF to FDCA, pair with hydrogen production, and PEF production as in claim 1, wherein after the reaction, due to the insolubility of DPhSO in water and the high partition coefficient of HMF in the DPhSO/water, the HMF product can be simply collected by extraction and the obtained HMF solution can be directly fed to the flow-cell electrolyzer with KHCO₃

12. The method of electrocatalytic production of polyethylene furanoate degradable bioplastic comprising of dehydrogenation of fructose to produce HMF, the near-neutral electro-oxidation of HMF to FDCA, pair with hydrogen production, and PEF production as in claim 1, wherein with the in-situ alkaline modification (by KOH), the HMF solution flow is converted into a flow of FDCA solution under applied potential and then the FDCA solution is acidified by HCl and thus the FDCA precipitates as a solid, which is subsequently separated and collected as white powder.

13. The method of electrocatalytic production of polyethylene furanoate degradable bioplastic comprising of dehydrogenation of fructose to produce HMF, the near-neutral electro-oxidation of HMF to FDCA, pair with hydrogen production, and PEF production as in claim 1, wherein the obtained FDCA is used as feedstock to produce PEF bioplastic via a two-step condensation polymerization.

14. The method of electrocatalytic production of polyethylene furanoate degradable bioplastic comprising of dehydrogenation of fructose to produce HMF, the near-neutral electro-oxidation of HMF to FDCA, pair with hydrogen production, and PEF production as in claim 1, wherein the final product of PEF is presented in a form of plastic pellet.
